(19) United States
(12) Patent Application Publication
(10) Pub. No.: US 2003/0004315 A1
(43) Pub. Date: Jan. 2, 2003

(54) Collagen

(76) Inventors: Grant Arthur Macdonald, Nelson (NZ); Kathleen Hofman, Nelson (NZ)

Correspondence Address: JACOBSON HOLMAN PLLC, 400 SEVENTH STREET N.W., SUITE 600, WASHINGTON, DC, 20004, US

(21) Appl. No.: 10/129,129

(22) PCT Filed: Nov. 29, 2000
(86) PCT No.: PCT/NZ00/00237

(30) Foreign Application Priority Data
Nov. 29, 1999 (NZ) 501386

(51) Int. Cl. C07K 14/78

(52) U.S. Cl. 530/356; 106/160.1

(57) ABSTRACT

The present invention relates to novel collagen products, methods for producing same, and to a novel test for assessing native collagen content of a protein solution. Particular collagen products are extracted from the skin of cold water fish. The collagen products comprise a high proportion of native collagen and are suitable for use as fining agents in the brewing and wine industries.

This invention relates to collagen products and to methods for producing such products. In particular, it relates to collagen products extracted from the skin of cold water fish, suitable for use inter alia as a fining agent in the brewing and wine industries. Also provided is an assay for determining the amount of native collagen present in a protein sample.

### BACKGROUND

Collagen products have a number of applications in various industries. In one such application, collagen finings are used in clarification or precipitation processes, for example for clarifying potable liquors such as beer and wine. During the fermentation of liquors various particulate materials such as yeasts and proteins become suspended in the liquor and need to be removed. Collagen finings are added to the liquor to clarify it by aiding the precipitation of the suspended materials.

Collagen finings are generally prepared from fish isinglass, which constitutes a very pure source of collagen prepared from the dried swim bladders of fish. The properties of such finings reflect their swim bladder derivation and the fact that the swim bladders are extracted from warm water fish such as members of the polynemidae family^{2,3,4}.

Another source of collagen is the skin. Many investigations have been made into the extraction of collagen from animal and fish skins including cold water fish skins (U.S. Pat. Nos. 4,295,894, 5,698,228, 5,162,506, 5,420,248, JP 4037679, JP 9-278639, JP 2-291814, PL 312122, RU 2139937). The collagen extraction processes known involve a wide range of chemical and mechanical extractions, or combinations thereof. The properties of the collagen products obtained by these processes vary widely. Many of the extraction processes applied to fish skins are adaptions of mammalian collagen extraction techniques. The applicants have identified that many of the processing steps applied to mammalian collagen extraction are not necessary for fish skin collagen extraction. Such steps including chemical washes and extractions, filtering, vacuum filtration, and decantation steps, and enzyme extractions amongst others. A simplified extraction process which eliminates many of these steps would be desirable.

It has also generally been accepted that collagen produced from skins of cold and deep water fish exhibits variable quality and is generally inferior to collagen produced from isinglass in finings production. It has therefore been concluded that cold and deep water fish skin collagen is unsuitable as a substitute for isinglass.¹

The applicants have now surprisingly determined that it is possible to extract collagen from fish skin, specifically the skin of cold water fish, which collagen when extracted is suitable for use as a fining agent. Furthermore, the applicant's collagen has different and advantageous properties as compared to collagen finings from isinglass.

The present invention therefore provides collagen products which are, inter alia, suitable for use as finings.

It is therefore an object of this invention to provide a collagen product suitable for use as a fining agent, or at least to provide the public with a useful choice.

It is a further object of this invention to provide a simplified or alternate method for collagen extraction from cold water fish skins.

Collagen is recognised as a difficult and expensive protein to quantify because of the insoluble nature of most collagens. Yet, solubility is a key functional property important in a variety of applications such as healthcare products⁵. It has been determined that the conformation of the native collagen molecule determines molecular functionality, with transition to the random coiled confirmation of gelatin upon denaturing resulting in a significant loss in fining ability.

A number of methods are known for determining collagen concentration. These include determining hydroxyproline (unique to this protein) content⁶, sophisticated gel electrophoresis and/or HPLC (requiring sample solubilisation) tests, kjeldahl for nitrogen, and polarimetry methods⁷. These methods are all complex and time consuming and require sophisticated and/or expensive equipment. It would therefore be a significant advantage to have a simple method for determining the amount of native collagen in a protein sample.

The applicants have unexpectedly found that total content of collagen in a sample from skins of cold water fish can be measured using the biuret method (Gornall et al.)⁹ and that native collagen content can be quantified using dyes which bind only to native collagen. These assays taken together provide a simple, rapid method for determining native collagen concentration in a sample.

Accordingly, it is a further aspect of this invention to provide a method for determining native collagen concentration in a sample, or again at least to provide the public with a useful choice.

### SUMMARY OF THE INVENTION

In one embodiment, the invention provides a fish skin collagen product in which native collagen forms 75% or more of the total protein content as measured by the Hofman test, which has an imino acid content of less than 170 per 1000 amino acid residues, and which meets, in addition, at least one of the following requirements:

(a) in solutions of about 6 mg/mL total solids dissolved in water, more than 60% is protein recoverable from the supernatant after centrifuging at 40,000× g for 30 minutes at 5° C.; and

(b) the intrinsic viscosity will be between 13 and 30 dL/g determined in 0.1 M acetic acid at 10° C.

By “native collagen”, it is meant the tripeptide tropocollagen, consisting of three α-chains, substantially in its native soluble form.

Conveniently, more than 60% of the protein remains in the supernatant after centrifuging a range of solutions of between 2-10 mg/mL total solids dissolved in water at 40,000× g for 30 minutes at 5° C.

In a further embodiment, the invention provides a collagen product obtainable by stabilising a collagen paste by the addition of a preservative, said paste being the result of the following pre-stabilisation steps:

(i) without a prior extraction, effecting swelling of skins from cold water fish from which all residual flesh, fats and pigments have been removed, said swelling involving contacting said skins for an appropriate length of time with an acid solution; and

(ii) without drying said swollen skins, homogenising said skins to form a paste

each of which steps is conducted at a temperature below the thermal melting temperature (Tm) for the cold water fish species from which the skins are obtained.

As used herein, “cold water fish” means a fish having a skin containing protein in which the imino acid content is less than 170 per 1000 amino acid residues and/or the Tm is lower than 20° C. Examples of such fish include New Zealand hoki (also called blue grenadier), Atlantic cod, Alaska pollock, arrowtooth flounder, Pacific whiting, South African hake, South American hake, South American hoki, lingcod, and salmon.

Preferably, said skins in step (i) are undried.

Preferably, all residual flesh, fat and pigments are removed from said fish skin by a mechanical cleaning process such as an abrading or scraping process. It is particularly preferred that all residual flesh, fat and pigments be removed using tumble abrasion in water, or with water jets.

Conveniently, said skins are contacted with said acid solution of less than 0.5 M concentration for at least 30 minutes.

It is preferred that the acid be selected from acetic acid, citric acid and tartaric acid although inorganic acids may also be used.

Preferably, said skins are contacted with said acid solution at a ratio of from 10-200 g of skin per liter of acid.

Preferably, in said process the homogenised skins, in the form of a paste, are allowed to stand for at least 30 min at a temperature of less than 15° C., and desirably less than 12° C. prior to stabilisation.

Preferably, said preservative is a food growth preservative such as a sulphite, an antimicrobial organic acid or a salt thereof (eg. sorbate, acetic and lactic acids).

Said stabilised paste is either stored at a temperature of 12° C. or less or is dried.

Generally described is a process for the extraction of protein from fish skin wherein said protein is predominantly native collagen comprising providing a starting material consisting essentially of skin from cold water fish from which all flesh, fat and pigments have been removed, extracting protein from said fish skin and recovering said protein.

Preferably, said skins are undried.

In still a further embodiment, the invention provides a method for preparing a stabilised collagen paste which comprises the step of adding a preservative to a collagen paste, said paste having been obtained by the following pre-stabilisation step

(i) without a prior extraction, effecting swelling of skins from cold water fish from which all residual flesh, fats and pigments have been removed, said swelling involving contacting said skin for an appropriate length of time with an acid solution; and

(ii) without drying said swollen skins, homogenising said skins to form a paste,

each of which steps is conducted at a temperature below the Tm of the cold water fish from which the skins are obtained.

The invention also provides a method for preparaing a pre-stabilised collagen paste which comprises the steps of:

(i) without a prior extraction, effecting swelling of skins from cold water fish from which all residual flesh, fats and pigments have been removed, said swelling involving contacting said skin for an appropriate length of time with an acid solution; and

(ii) without drying said swollen skins, homogenising said skins to form a paste,

each of which steps is conducted at a temperature below the Tm of the cold water fish from which the skins are obtained.

Preferably, said skins are undried.

It is also preferred that swelling is effected using an organic acid, for at least 30 minutes.

In still a further embodiment, the present invention provides a method for preparing a collagen product in which at least 75% of the total protein is in the form of native collagen, said process comprising the steps of

(i) removing all residual flesh, fat and pigments from fish skins by a mechanical cleaning process;

(ii) without a prior extraction, effecting swelling of said skins through contacting said skins with an acid solution for an appropriate length of time;

(iii) homogenising said swollen skins to form a paste;

(iv) holding the resulting paste for an appropriate period of time to allow the collagen present to complete swelling and solubilisation; and optionally

(v) adding a preservative to stabilise said paste.

In an alternate process step, (iii) may be effected prior to step (ii).

Preferably, in step (iv), said paste is held for a minimum of 30 minutes.

Preferably, said method further includes the step of:

(vi) drying the stabilised paste at a temperature below the Tm of the fish from which the skins were obtained.

In yet a further embodiment, the present invention provides a collagen product which is prepared by a process as defined above.

In one embodiment, the collagen product is in the form of a stabilised paste.

In an alternative embodiment, said collagen product is a dried product.

In still a further embodiment, the invention provides a composition which includes a collagen product as defined above.

In one embodiment, said composition is a foodstuff.

In another embodiment said composition is a fining agent.

In an alternative embodiment, said composition is a medicament, nutriceutical or cosmetic.

In still a further aspect, the invention provides a method of clarifying potable liquor to remove particulate matter present in said liquor which comprises the step of adding a collagen product as defined above to said liquor.

In a yet further aspect, the invention provides a method for determining the native soluble collagen content of a protein solution, the method comprising:

(a) determining the total protein content of the solution using a biuret assay;

(b) determining the amount of native collagen in the solution using a Sirius red dye binding assay; and

(c) comparing the results of (a) and (b) to give the proportion of native collagen in the protein solution; and

wherein step (b) is carried out at temperature less than the thermal melting temperature of the collagen being tested for.

In a still further aspect, the invention provides a kit for determining the amount of native collagen in a sample, the kit comprising copper sulphate, potassium sodium tartrate, and Sirius red.

### DESCRIPTION OF THE DRAWINGS

While the present invention is broadly as defined above, it will be appreciated by those persons skilled in the art that it is not limited thereto but that it also includes embodiments of which the following description provides examples. In addition, a better understanding of the invention will be gained through reference to the accompanying drawings in which:

FIG. 1 is a graph showing reduced viscosity versus concentration of freeze-dried hoki skin collagen in 0.1 M acetic acid determined at 10° C. The intercept of the vertical axis is the intrinsic viscosity for this hoki skin collagen, a value of 20.4 dL/g.

FIG. 2 is a plot of viscosity ratio versus temperature for a 1 mg/mL hoki skin collagen solution in 0.5 M acetic acid. Solutions were held at the indicated temperature for 30 min prior to measurement in an Ubbelohde-type capillary viscometer (model #1, Cannon Instrument Co, PA, USA).

FIG. 3 shows the solubility of hoki skin collagen in water, 0.1M acetic acid and 0.1M citric acid after solubilisation of the dry material (Before) and after centrifigation at 40,000× g for 30 min at 5° C. (After Centrifugation).

FIG. 4 shows the fining effectiveness for chardonnay wine shown by a decrease in absorbance at 280 nm with increasing concentration of soluble hoki skin collagen. (a) Three hoki collagen preparations are shown freeze-dried material solubilised in 0.1 M citric acid and paste solubilised in both 0.1 M citric and 0.1 M acetic acid. (b) Freeze-dried hoki skin collagen solubilised in chilled 0.1 M citric acid at a concentration of 10 mg/mL prior to final dilution in the chilled wine.

FIG. 5 shows L to R control (isinglass 25 mg/L), hoki skin collagen (10 mg/L) and untreated beer.

FIG. 6 shows L to R Control (isinglass 20 mg/L), untreated wort, hoki skin collagen (20 mg/L).

FIG. 7 shows fining ability of hoki skin collagen compared to a commercial isinglass product (Control). Hoki skin collagen was added to unfined beer at 12 mg/L and the Control was added at 22 mg/L.

FIG. 8 shows comparison of clarity and stability of beer fined with either hoki skin collagen or a commercial isinglass preparation (Control). Haze at 90° and 25° forward scatter (EBC units) after 1 month and 2 month's storage. Hoki skin collagen was added to unfined beer at 12 mg/L and the Control was added at 22 mg/L.

FIG. 9 shows sensory analysis of chardonnay wine from fining trials using four fining agents including hoki skin collagen. Shows increased fruitiness and reduced astringency when hoki skin collagen was used as a fining agent (0.03 g/L concentration hoki skin collagen and stored for 96 hr at 10° C. prior to analysis).

FIG. 10. Standard curve for biuret method of protein estimation using hoki skin collagen disolved in 0.1 M acetic acid.

FIG. 11. (a) The precipitation of the dye Sirius Red by hoki skin collagen at 10° C. (□), as shown by a decrease in absorbance at 540 nm. (b) Loss of ability to quantitatively precipitate the dye after hoki collagen was heated to 30° C. for 10 min prior to assay (⋄).

FIG. 12. (a) Concentration of hoki collagen in solution with change in temperature (□) as measured by the binding of Sirius Red F3BA dye. Each sample point is the average of five dye assays. (b) The change in viscosity ratio with temperature (⋄) for a 1 mg/ml hoki collagen solution in 0.5M acetic acid. Each sample point is the average of three viscosity measurements.

### DESCRIPTION OF THE INVENTION

As broadly defined above, the primary focus on the present invention is on a collagen product. This collagen product has application in the liquor, food, cosmetic and healthcare industries.

The essential finding of the applicants is that it is possible to extract collagen from the skins of cold water fish in a manner such that the resulting extracted collagen has advantageous properties. Those properties make the resulting collagen particularly suitable for a number of applications, including use in the clarification of potable liquors such as beer and wine.

Examples of cold water fish from which skin can be obtained for use as the starting material for the extraction process of the invention include New Zealand hoki (also called blue grenadier), Atlantic cod, Alaska pollock, arrowtooth flounder, Pacific whiting, South African hake, South American hake, South American hoki, lingcod, and salmon. However, this list is by no means exhaustive and should not be interpreted as limiting.

One primary quality of the collagen of the invention is that at least 75% of the total protein present is native collagen. The percentage of native collagen present is measured by the Hofman test.

By the “Hofman test” it is meant a novel combination of two assays for determining the relative proportion of a protein solution that is made up of native collagen. Specifically, total protein in the solution is determined by the biuret reaction which uses a copper complex to measure the number of peptide bonds, whereas native collagen is determined by the ability of the dye Sirius Red to quantitatively bind to native collagen molecules and cause the collagen molecules to precipitate from solution.

The applicants have determined that the biuret reaction will give a linear standard curve up to a concentration of 6 mg/mL using freeze-dried fish skin collagen. Briefly, 0.5 mL of sample (1-5 mg/mL protein) is added to 2.5 mL biuret reagent (0.006 M copper sulphate, 0.02 M potassium sodium tatrate in strong NaOH solution) mixed and the absorbance at 540 nm is read after 20 min. The absorbance is calibrated against a standard curve (0-5 mg/mL protein) using hoki skin collagen to give the total protein present.

The applicants have further determined that the dye binding assay using Sirius Red binds only to native collagen molecules, and can be used in the range 5 to 65 μg collagen. To 5-65 μg protein in 100 μL 0.5 M acetic acid is added 1.0 mL Sirius Red solution (50 μM Sirius Red in 0.5 M acetic acid). After 30 min samples are centrifuged at 11,000× g and the absorbance of the supernatant is read at 540 nm. The absorbance is calibrated against a standard curve using samples of 5-65 μg collagen in 0.5 M acetic acid.

Comparison of the results from the biuret and the dye binding assays gives the relative proportion of native collagen in the protein solution.

Dye binding is carried out at a temperature less than the thermal melting temperature (Tm) for that collagen. The Tm is a measure of the transition from soluble collagen molecules to denatured collagen chains or gelatin and is defined as the temperature at which half of the initial intrinsic viscosity disappears in 30 minutes.

Accordingly, the present applicants have been the first to provide teaching of a rapid, simple and reliable test for determining the proportion of native collagen in a protein solution, especially collagen from cold water fish skins. The Hofman test therefore forms a further aspect of this invention.

The Hofman test also forms the basis of a kit for the determination of native collagen in a protein sample. Accordingly, also provided by the invention is a kit suitable for use in the Hofman test. The kit comprises the assay reagents, namely copper sulphate, potassium sodium tartarate, and Sirius Red. The reagents may be made up in solution ready for use. The reagents are separately contained, and are provided in amounts sufficient for carrying out at least one, and preferably multiple Hofman tests. The kit may further comprise a standard for the collagen being tested.

In addition, the collagen product of the invention is defined by reference to functional criteria. These include solubility in water and intrinsic viscosity.

In terms of solubility, in solutions of between 2-10 mg/mL total solids dissolved in water, more than 60% is protein recoverable from the supernatant after, centrifuging at 40,000× g for 30 minutes at 5° C. Conveniently, the solubility measurement is made at a concentration of between 4-8 mg/mL and most usually at about 6 mg/mL in water, and protein recovery is more then 70%.

As for intrinsic viscosity, this will be between 13 and 30 dL/g determined in 0.1 M acetic acid at 10° C. using an Ubbelohde-type capillary viscometer (model #1, Cannon Instrument Co, PA, USA).

The invention also provides a collagen product obtainable by stabilising a collagen paste by the addition of preservative. The paste is pre-stabilised by swelling defleshed, defatted, depigmented cold water fish skins in acid solution. The defatting, defleshing and depigmentation is effected by mechanical cleaning processes known

in the art, including abrading and scraping. Preferably, flesh, fat and pigments are removed by tumble abrasion in water or using water jets.

Acid solutions may be inorganic acids such as hydrochloric or sulphuric acid, but are preferably organic acids such as acetic, citric and tartaric acid but are not limited thereto. Dilute acid solutions of from 0.05 to 0.8M are generally used. Preferably, 0.1 to 0.5M.

The skins are contacted with the acid solution at a ratio of from 10-200 g of skin per liter of acid. The ratio is dependent on the type of collagen product desired. More concentrated pastes (5-50 mg collagen/mL) are suitable for shipping and storage while dilute solutions (8 mg collagen/mL and lower) may be more convenient for actual use.

Swelling time is desirably at least 30 minutes, up to 1 or 2 days, or even weeks if the paste is to be stored prior to stabilisation.

In a preferred process the homogenised skins, in the form of a paste, are allowed to stand for at least 30 minutes, or for hours, days or weeks at a temperature less than 15° C., and most desirably less than 12° C.

Homogenisation is carried out using standard art techniques. All steps are conducted at a temperature below the thermal melting temperature for the cold water fish species from which the skins are obtained. This avoids denaturation and loss in functionality of the collagen product.

The preservative added may be any preservative suitable for use in foodstuffs. This includes sulphite, antimicrobial organic acids or salts thereof but are not limited thereto. Preferred preservatives are sulphite, sorbate, acetic acid and lactic acid.

The skins can be fresh, frozen or dried. In a currently preferred embodiment the skins are undried.

The solubilised paste is stored in the same conditions as the paste pre-stabilisation, or is dried. Drying may be carried out using known art techniques. Preferably freeze drying is carried out keeping heating temperatures below the Tm of the collagen.

In a related aspect, the invention also provides a method for preparing a stabilised collagen paste by adding a preservative to a collagen paste pre-stabilised according to the above methods.

This invention also provides a process for extracting protein from fish skin, which protein is predominantly native collagen. As discussed above, the starting material consists essentially of skin from cold water fish. This skin is subjected to flesh, fat and pigment removal as discussed. Protein extraction is then effected by swelling the skins with a dilute acid solution for an appropriate length of time.

Any of the acids referred to above may be used. Preferably, the organic acids are used. The extraction step is carried out for at least 30 minutes and usually for hours, or even several days. The protein solubilised in the acid is then recovered as a paste.

Again, the skins used in the process can be fresh, frozen or dried. In a currently preferred embodiment the skins are undried.

The collagen product of the invention is obtainable from cold water fish skins essentially in accordance with the following (preferred) process steps.

1. Raw material

Skins are obtained from the skinning operation from either fresh or frozen fish, alternatively frozen skins are thawed.

2. Cleaning

The skins are subjected to mechanical cleaning by abrasion or scraping in tap water. Residual flesh, fats and pigments are removed by scraping, in tumble abrasion or using water jets. The process is repeated until the skins are sufficiently clean. These skins may be used directly or frozen or dried for later manufacture.

3. Swelling

Clean skins are swollen for 30 minutes or more in dilute organic acid (acetic, citric, tartaric or other) at a ratio of 10-200 g per liter clean skin to acid.

4. Communition

Swollen skins are homogenised in a vertical cutter/mixer or similar machine to make a fine paste.

5. Extraction

The fine paste is held for a period of time to allow the collagen fibers to complete swelling and solubilising.

6. Storage

The collagen paste is stabilised with acceptable preservatives and stored at temperatures below 12° C. Preservatives that may be used are sulphite, antimicrobial organic acids or their salts (eg. sorbate, acetic and lactic acids) and other preservatives commonly used in the food industry.

The collagen product may be retained in the form of the stabilised paste or can be part of a solution. It is also possible to provide the collagen product in a dry form, in which case it is preferred that the process include the following step:

7. Freeze drying

Solubilised collagen is dried at temperatures less than the Tm of the fish species from which the skins were derived for sale as a dry product.

In step (1) the preferred mechanical cleaning process is water abrasion. Acid swelling of skins may also be effected using any of the acid solutions discussed above, and for the times given. In step (3) the paste is usually held for a minimum of 30 minutes and more usually hours or days.

The order of steps 3 and 4 may also be reversed. That is, communition may be effected prior to swelling.

A wide range of communition techniques and apparatus known in the art may be used as alternatives to the vertical cutter/mixers.

Extraction step 5 as noted above is carried out for at least 30 minutes, and preferably for hours or days to allow the collagen fibres to complete swelling and solubilising.

The optional freeze drying step is also effected using standard art techniques, desirably keeping heating temperatures below the Tm of the collagen.

The invention also relates to collagen products produced by the processes of the invention. The collagen products may be used as prepared, or may be incorporated in compositions including foodstuffs, such as hydrocolloids, or in medicaments, nutraceuticals or cosmetics. These applications for collagen are well documented in the art.

In a further aspect the invention also provides a method of clarifying potable liquor such as beer or wine to remove particulate matter. As with the use of known isinglass collagen products, collagen is mixed in with the liquor and allowed to settle. This removes yeast cells and/or other suspended components of the liquor. The collagen product or paste of the invention may be used. Preferably, the product is the paste in acid, or freeze dried collagen in acid. The collagen is added in an amount of from 0.005 to 0.5 g/L, preferably 0.01 to 0.4 g/L, and more preferably 0.02 to 0.1 g/L. For beer the collagen is most usually added in an amount of from 0.02 to 0.1 g/L. For wine the collagen is most usually added in an amount of from 0.01 to 0.04 g/L.

The collagen product of the invention is highly effective in clarifying both beer and wines. Stored beer products showed showed much lower haze levels than beer treated with current isinglass fining products. Compared to known isinglass products the collagen products of the present invention worked at least twice as fast, and removed more suspended product in beer and wort. In red wines colour was better using this product, while red and white wines showed softening of flavour, namely reduced astringency and unmasking of fruit flavour, retention of tannins for flavour structure, as well as good retention of volatile flavour components, and less lees production. Hoki skin collagen is therefore a useful fining agent.

The invention will now be illustrated with reference to the following non-limiting examples.

### EXAMPLE 1

Extraction of Collagen Product from Hoki Skin.

Fresh hoki were skinned using a Trio skinning machine (Trio, model FDS-2N skinner, Sweden). The skins were then added to a vertical cutter/mixer (Stephan Machinery, NZ) with a ratio of 1.5 kg skins to 1000 mL ice/water slurry. The mixture was comminuted for 1 min on speed setting one after which the skins were separated from the waste water by sieving. The skins were added back to the vertical cutter/mixer with 1000-2000 mL ice/water slurry and the tumble abrasion process was repeated for a total of 10 times. The cleaned skin tissue (110 g) was then added to 3000 mL of chilled O. 1M acetic acid and allowed to swell for 90 min. The mixture was then comminuted in the vertical cutter/mixer for 2 min on setting 1 to make a fine viscous paste of about 8 mg/mL collagen. The material was kept below 12° C. during all processing steps.

The resulting paste can be stored for up to 12 months under chill conditions (4° C.) prior to use or used directly in applications such as fining. For fining the paste is typically diluted prior to use to give a final concentration between 1 and 80 ppm depending on the final product attributes required.

Alternatively, the paste was freeze-dried to produce a dry, shelf stable material with a moisture content less than 12%. In this case paste was spread thinly on dryer trays, frozen to below −20° C. and freeze-dried for up to 48 hr. Low temperatures during drying were maintained by circulating tap water (18-20° C.) to heat the dryer trays. Typically the product temperature was maintained below 0° C. for the constant drying rate period which finished about 12 hr from the start of drying. Following completion of drying product was removed from the trays by scraping. The dry product was then packed into heat sealable aluminium laminate bags for long term storage. A yield of 1.5 g dry protein from 10 g wet cleaned skins can be attained with the resultant product having greater than 75% native collagen as determined by the Hofman test.

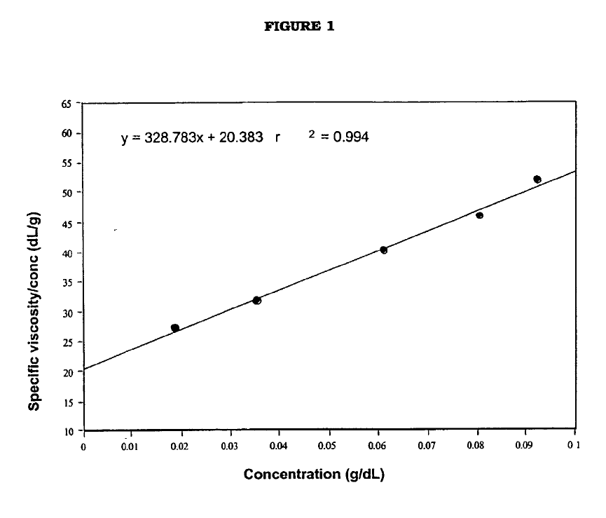

FIG. 1 shows results to determine the intrinsic viscosity of freeze-dried extracted hoki skin collagen. For this material the intrinsic viscosity in 0.1 M acetic acid was 20.4 dL/g as shown by the intercept of the reduced viscosity versus concentration plot (FIG. 1).

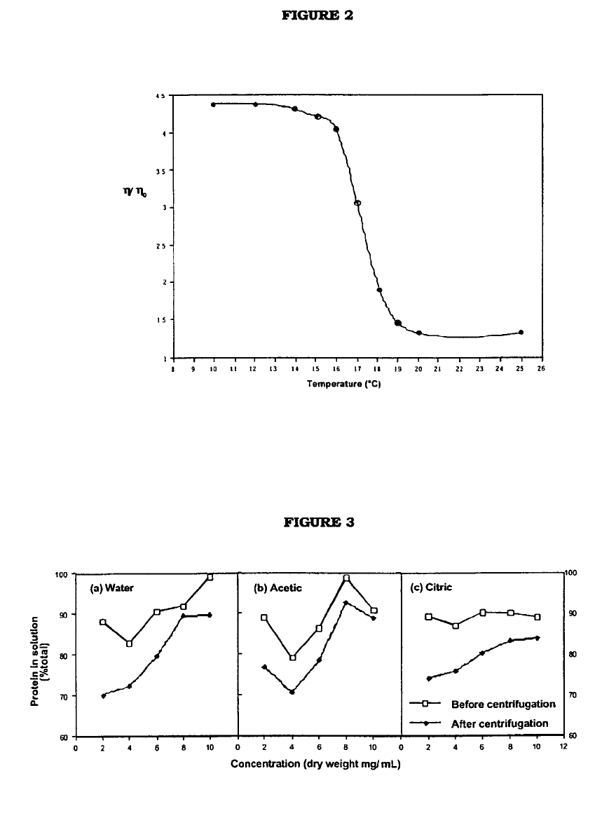

FIG. 2 shows results for determining the Tm, thermal melting temperature, of 17.2° C. for hoki skin collagen. The Tm is a measure of the transition from soluble collagen molecules to denatured collagen chains or gelatin and is defined as the temperature at which half of the initial intrinsic viscosity disappears in 30 minutes.

FIG. 3 shows the results of solubility studies demonstrating the highly soluble nature of hoki skin collagen in water and in dilute organic acid solutions. For example, after centrifugation to remove colloidal particles, and over the concentration range 2-10 mg/mL added protein, more than 70% of the protein remained dissolved in water. Similar results were gained when 0.1M acetic or citric acid were the solvent.

Briefly, freeze-dried protein extracted from hoki skin (as described herein) was mixed with 100 mL solution and stirred for 60 min (8° C.). The mixture was then homogenised with an Ultraturex mixing tool (IKA-Laboratechnik, model T25, Germany) using two 10 see bursts at 13,500 rpm. The solutions were shaken for a further 48 hr (8° C.), after which the total protein concentration was determined by biuret. The solutions were centrifuged for 30 min at 40,000× g (5° C.) and the protein content of the supernatant was measured by biuret to determine the amount of protein remaining in solution.

### EXAMPLE 2

Use of Hoki Skin Collagen as a Fining Agent in Wine

A stock solution of 5 mg/mL hoki skin collagen in chilled 0.1 M citric or acetic acid was made by stirring until the paste or freeze-dried material was dissolved. Aliquots of the stock solution were then added to 50 mL prechilled chardonnay wine in a 50 mL measuring cylinder to give a final concentration between 0 (control) and 60 ppm. Sufficient chilled water was also added to give a constant dilution for all treatments. The measuring cylinder was covered with Parafilm and gently inverted to mix the fining agent and wine and then stored at 8° C. for 24 hr. The clarified wine was then filtered through no. 1 Whatman filter paper, diluted 1:10 with distilled water and the absorbance at 280 nm was determined in a spectrophotometer (Univam, model UV4-100) using water as a blank.

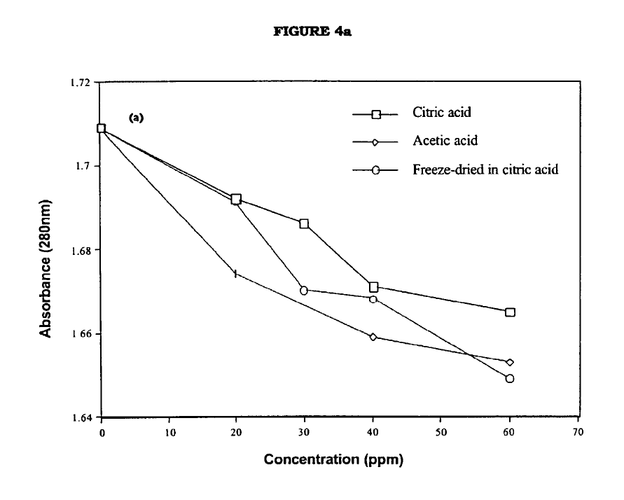

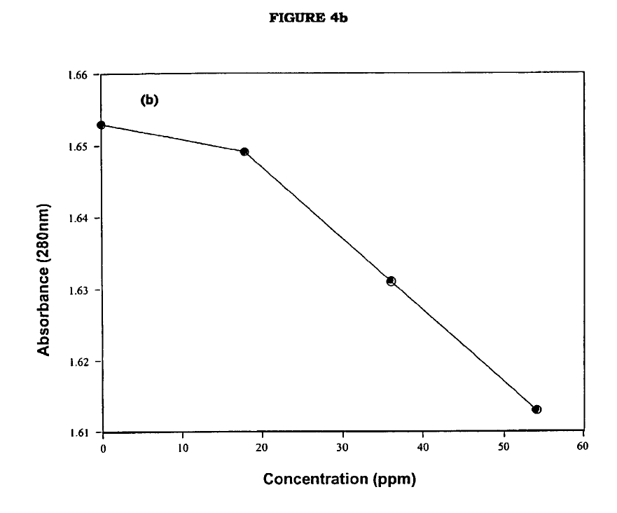

FIGS. 4 ((a) and (b)) demonstrates the effectiveness with increasing concentration of two forms of hoki skin collagen (paste dissolved in acetic and citric acid; and freeze-dried collagen in citric acid) for removal of phenolics and proteins associated with turbidity and bitter or astringent flavours in white wine.

### EXAMPLE 3

Use of Hoki Skin Collagen as a Fining Agent in Brewing

Introduction

Laboratory scale trials and an initial trial in 50 L kegs were carried out using a collagen product of the invention with potential use in the fining of beer and wine. The product was presented in a solid form and as a paste. These trials showed good fining properties. The trials were repeated on a 50 L basis to judge the effect on other beer parameters such as taste, head retention and haze stability.

Summary

Trial products were added to unfined beer at 12 mg/L and performance compared to a control of current commercial products sourced from Biocon (Quest International, NZ) and AB Vickers Ltd (UK) added at the current rate. The collagen product of the invention was found to be an effective fining agent.

Results

Results of Studies Using Hoki Skin Collagen in Brewing

After laboratory and industrial trials, hoki skin collagen has been found to be a highly effective fining agent for beer and wort. Hoki skin collagen is more effective than some commercially available isinglass-based preparations. In addition, hoki skin collagen confers excellent chill-proofing capability, haze stability on storage and has no effect on taste and head retention of beer.

Ability to Fine

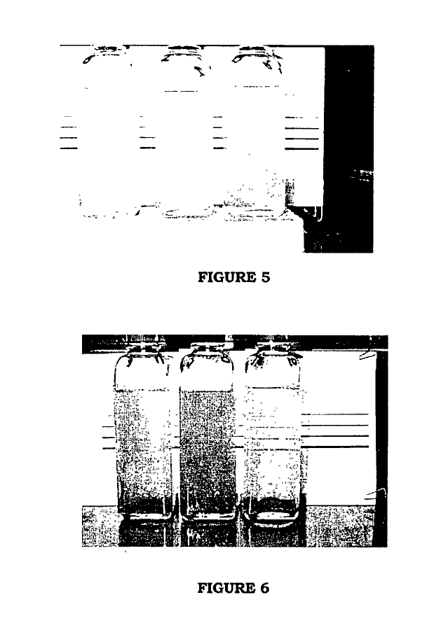

FIGS. 5 and 6 show the excellent fining ability of hoki skin collagen compared to the commercially available isinglass preparation (Control). In these small-scale trials hoki skin collagen settled within 24 hr while the Control sample of beer and wort took approximately twice as long to clear. For beer the dosage rate for hoki skin collagen was less than half that needed for the Control.

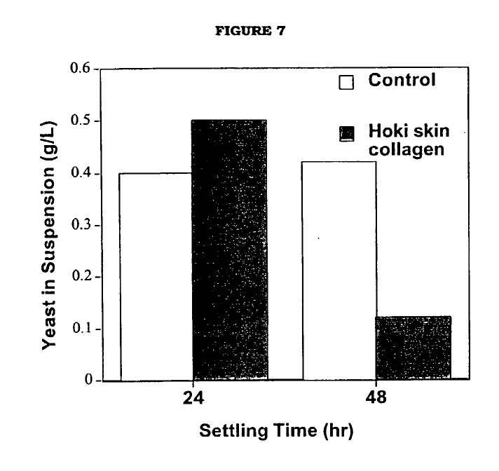

FIG. 7 shows the results from larger scale trials in which hoki skin collagen treated beer cleared faster and removed much more suspended yeast than the commercial isinglass-based product.

Clarity and Stability

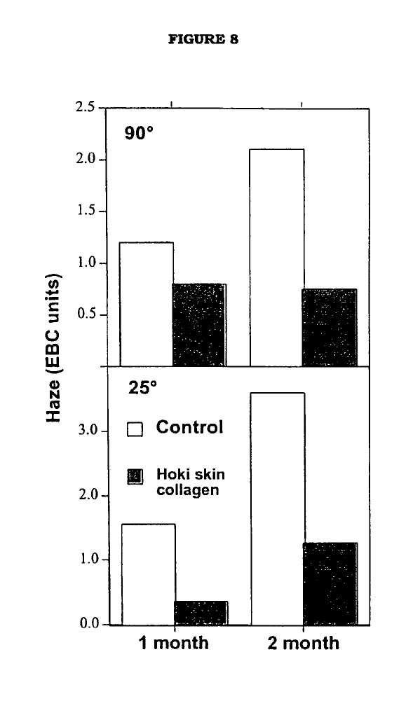

FIG. 8 shows results of a storage trial in which hoki skin collagen treated beer was clearer and more stable during storage compared to the Control. After 2 months storage hoki skin collagen treated beer had a haze reading (90°) about 70% lower than for the Control treated beer.

### EXAMPLE 4

Use of Hoki Skin Collagen as a Fining Agent in White and Red Wine

In white wines isinglass has been linked to a reduction in browning potential. It achieves the above without unduly decreasing the tannin concentration, or affecting the colour of the wine. The result is a brilliant, clear and softer wine. Isinglass is typically added at a rate of 0.02 to 0.1 g/L (20-100 ppm) producing compact lees of approx. 2%.

Herein we show experimental results for use of hoki skin collagen as a fining agent in white and red wine. Hoki skin collagen performs similarly to commercially available isinglass-based preparations. However, because of its improved functional properties hoki skin collagen has additional benefits associated with it including less loss of colour in light red wines such as Pinot noir, softening of flavour in white and red wines, and retention of tannins for flavour structure.

Collagens from skin of cold-water fish species provide further advantages for fining of wines made at low temperatures to retain volatile flavour components associated with aromatic wines such as sauvignon blanc, reisling and gwertztraminer.

Experimental

Pinot noir wine was from a barrel sample. A stock solution of 0.6% w/v was made up of freeze-dried hoki skin collagen by adding 0.2 g dry material to 33 mL deionised water. This was stirred for 2 min, left for 15 min to swell, and then stirred again to give a clear solution with no visible clumps. Treatments were performed in duplicate. Stock solution was slowly dispensed into 100 mL wine in a 100 mL measuring cylinder that had been sparged with CO₂ to give the following final concentrations; 0, 0.01, 0.02, 0.03, 0.04 and 0.1 g/L. The 0.1 g/L concentration was used to give an indication of the consequences of overfining with the agent. The treatments were then covered with Parafilm and mixed by gentle inversion 5 times, then left undisturbed in a 15° C. temperature controlled room for 48 hr.

The treatments were then carefully decanted off their lees into two 50 mL centrifuge tubes and spun at 4500 rpm, at −10° C. for 10 min. Samples were then vacuum filtered through 0.45 μm filters (Millipore Corp., USA). Sets of 10 mL samples were retained for specrophotometric analysis.

Spectrophotometric Analysis

Spectrophotometric analysis of wine colour parameters (A_{420 nm}; a measure of “brown” hue, A_{520 nm}; a measure of “red” hue, colour density; A_{420 nm}+A_{520 nm}, and colour hue; A_{420 nm}/A_{520 nm}, and of total phenolics; A_{280 nm}−4. All analyses were performed on a Unicam UV4-100 spectrophotometer controlled by Vision v3.10 software (Unicam, UK).

Results

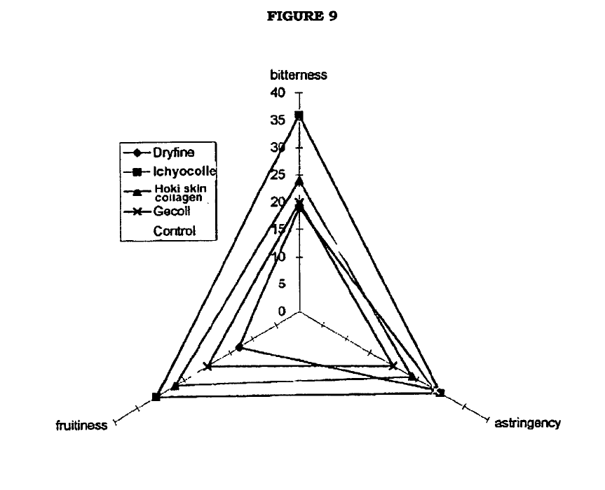

Hoki skin collagen produced a light “fluffy” lees layer that was easily disturbed but compact after 36 hr at 15° C. This layer was more compact than those produced by commercial isinglass preparations (refer FIG. 9) under the same conditions. The actual depth of the lees layer was related to the amount of agent added with about 7% percent lees produced when 0.04 g/L hoki skin collagen was added. Observation of the wine post settling indicated the fined wine had an increased clarity (“brightness”).

Effect of Fining with Hoki Skin Collagen on Wine Colour Parameters and Total Phenolics:

Table 1 shows that treatment with hoki skin collagen did not significantly alter wine colour parameters and total phenolics when the material was used within typical/practical limits. That is, when an application range between 0.01 and 0.04 g/L was used for clarification. The greater reduction in absorbance at 420 nm and 520 nm with the addition of 0.1 g/L shows that hoki skin collagen can alter phenolic composition and colour of the wine at higher use levels. This may be of benefit when a wine maker may wish to reduce “browning” in a wine, even at the expense of a reduction in some of the “red” hue. Similarly, higher concentrations of hoki skin collagen may be used to remove phenolics contributing to undesirable levels of bitterness and astringency. However, colour hue and total phenol content of the wine was not significantly altered by any of the addition rates of hoki skin collagen tested. hoki skin collagen is a useful fining agent, particularly with Pinot noir wine.

TABLE 1
Effect of hoki skin collagen treatments on Pinot noir wine

| Protein | | | | | |
|---|---|---|---|---|---|
| Conc | | | Colour | Colour | Total |
| (g/L) | A₄₂₀ₙₘ | A₅₂₀ₙₘ | Density | Hue | Phenolics |
| 0 | 1.94 | 2.28 | 4.22 | 0.854 | 4.29 |
| 0.01 | 1.92 | 2.24 | 4.17 | 0.857 | 3.65 |
| 0.02 | 1.91 | 2.22 | 4.13 | 0.863 | 3.98 |
| 0.03 | 1.92 | 2.24 | 4.17 | 0.857 | 4.63 |
| 0.04 | 1.92 | 2.23 | 4.15 | 0.859 | 3.91 |
| 0.1 | 1.80 | 2.09 | 3.89 | 0.858 | 3.67 |

### EXAMPLE 5

Application of Hofman Test to Hoki Skin Collagen

Introduction

Solubility of collagen is a key functional property that is important in a variety of applications. Collagens extracted from cold-water fish species are not only more labile but also have interesting functional properties, such as high solubility in dilute acid and increased ability to interact with colloidal particles.

The performance of finings has been shown to be related to the amount and size of the collagen molecules in solution (Leach and Barrett 1967)⁴. The conformation of the native collagen molecule determines molecular functionality since the transition to the random coiled conformation of gelatin results in significant loss in fining ability. Therefore, it would be a significant advantage to have a simple testing method to not only measure the amount of soluble collagen in a solution but also to quantitate the amount of native collagen, the more functional state.

Further, it has been found that collagen solubility is an important functional property for use in a range of healthcare products. For example, the use of collagen sponges as a matrix for bone or cartilage remodelling, for slow release of drugs or as a wound dressing (Chvapil 1979)⁵.

The insoluble nature of most collagens makes collagen a difficult protein to quantify. The methods that are used to determine collagen concentration are expensive and time consuming. These include, determining hydroxyproline content, kjeldahl for nitrogen, gel electrophoresis, chromatography and polarimetry methods. There is, therefore, a need for a simple, rapid method for determining total collagen and native collagen concentrations.

Experimental

Collagen Preparation:

Hoki skins were scraped clean of all adhering tissue and scales and washed in distilled water. The skin was then swollen in 0.1 M acetic acid in a ratio of 1:42 (g/mL), homogenised in a food processor (Magimix, Australia), and held for 18 hr at 8° C. The extracted material was then centrifuged for 60 min at 10,000× g (5° C.). The supernatant was diluted and taken to 1.0 M NaCl with the addition of 4.4 M NaCl solution. Following centrifugation for 60 min at 8,000× g (5° C.) the pellet was dissolved in 0.1M acetic acid and dialysed extensively before freeze drying for storage.

Total Protein Assay:

Total protein concentrations were measured according to the biuret method (Gornall et al., 1949)⁹ using hoki skin collagen as standard. Briefly, 1.5 g copper sulphate and 6.0 g sodium potassium tartrate was dissolved in 500 mL water. Then 300 mL of 10% sodium hydroxide was added and the solution was made up to 1 L with water to make the biuret reagent. To 2.5 mL biuret reagent was added 0.5 mL protein sample, the mixture was vortexed and held for 20 min at room temperature before determining the absorbance at 540 nm.

Native Collagen Assay:

The ability of the dye Sirius Red F3BA to quantitatively precipitate collagen in acid solution was used to determine the concentration of native collagen in a solution (Marotta and Martino 1985)⁸. Native collagen was assayed by adding 100 μL protein solution (5-65 μg protein in 0.5 M acetic acid) to 1.0 mL Sirius Red F3BA dye (50 μM in 0.5 M acetic acid). After 30 min the samples were centrifuged (11,600× g for 8 min) to remove precipitated collagen and the absorbance of the supernatant was determined at 540 nm.

Collagen Thermal Denaturation Studies:

Hoki skin collagen (0.7 mg/mL) was dissolved in 0.5 M acetic acid and samples were held for 30 min at temperatures from 10-23° C. After heating, samples were rapidly cooled in an ice slury and the concentration of the native collagen present was determined by the ability of the protein to bind Sirius Red F3BA dye as described above.

To compare the dye binding assay to another standard measurement we measured the change in viscosity as hoki skin collagen (1 mg/mL in 0.5 M acetic acid) was heated for 30 min at temperatures from 10-25° C. Viscosity was measured using an Ubbelohde-type capillary viscometer (model #1, Cannon Instrument Co, PA, USA).

Results

Biuret for Determining Total Protein

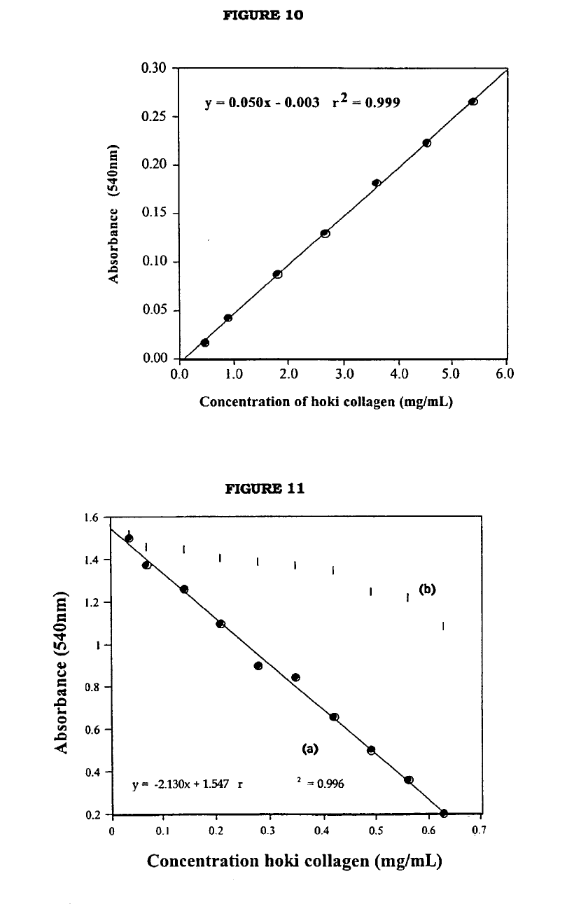

We used the biuret method for determining total collagen (or total protein) concentration of hoki skin collagen solutions over the range 0-6 mg/ml. FIG. 10 shows that hoki skin collagen behaves linearly with the biuret method of protein determination for concentrations up to 6 mg/ml. The biuret method was not affected by pretreatment temperatures that denatured all hoki collagen present in solution.

Dye-Binding Method for Determining Native Collagen

FIG. 11a shows the precipitation of the dye Sirius Red by hoki skin collagen when collagen solutions are stored and assayed below the collagen Tm. FIG. 11b shows the loss of dye binding ability when the hoki collagen was heated at 30° C. for 10 min prior to assay.

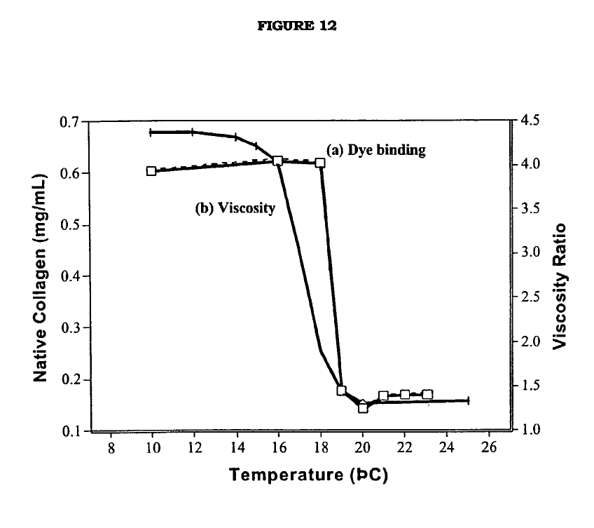

FIG. 12 shows the change in native collagen concentration as measured by dye binding (a) and viscosity (b) after heating at the indicated temperature for 30 min. The dye binding method followed changes in viscosity as the collagen molecules denatured and changed from a rod shape to a more globular structure. Dye binding was slightly less sensitive than the viscometric method to initial molecular shape changes in the initial part of the collagen to gelatin transition. This is demonstrated by an abrupt transition between 18 and 19° C. compared to the more gradual change in viscosity from 16 to 20° C. The transition temperatures derived from each method are close with 18.5° C. and 17.3° C. for dye and viscosity respectively.

Method for Determining Percentage of Total Protein That is Native Collagen

The dye Sirius Red F3BA only binds to native collagen molecules and will not bind to denatured collagen molecules. Therefore, a combination of the biuret and dye-binding methods gives specific information on the total quantity of collagen (or total protein) in solution and quantity that is in the native state. Conversely, the percent collagen that has been denatured can also be determined. This is valuable information for a number of industries that use collagen for its functional properties.

We have named this combined test the “Hofman Test”

In the fining industry the rapid and simple Hofman Test will make it possible to establish an industry standard test because of the difficulty of determining collagen content in products currently marketed and the lack of consistency with which this type of information is supplied by the producers of isinglass products.

### INDUSTRIAL APPLICATION

Thus, in accordance with the present invention there is provided a collagen product which possesses a range of advantageous functional properties. These properties reflect the derivation of the collagen from the skin of cold water fish and also the process of extraction.

The resulting collagen product has application in numerous industries. These include the food industry, the healthcare industry and the cosmetic industry.

One specific application of the collagen product of the invention is in the clarifying of potable liquors, particularly beer and wine. The applicants have found that their collagen product is able to be used as a fining agent. Furthermore, the results achieved are equal to if not better than those achieved using the traditional isinglass fining agents.

The invention also provides a collagen extraction process adapted for use on cold water fish skins. This process eliminates many unnecessary processing steps previously considered essential or important for mammalian and/or fish skins. The process is therefore simple and easy to carry out.

The Hofman test and related kits also make it possible to monitor an extraction process, to develop effective purification procedures, to monitor samples during storage, and to develop a collagen product to a required specification.

It will be appreciated by those persons skilled in the art that the above description is provided by way of illustration only and that changes and modifications, particularly to the preparative process, may be made without departing from the scope of protection claimed.

### REFERENCES

1. Anderson, R. G., Martin, P. A. (1979) An investigation of Finings from Fish skins. J. Inst. Brew. 85: pp 240-242.

2. Vickers, J. (1985) Isinglass in wine. Tech. Bulletin, Isinglass factory. Coggeshall, Colchester, Essex, UK.

3. Leach, A. A. (1967) Collagen Chemistry in relation to Isinglass and isinglass finings—a review. J. Inst. Brew. 73: pp 8-16.

4. Leach, A. A. and Barrett, J. (1967) The Molecular weight and soluble collagen content of finings in relation to its fining potential. J. Inst. Brew. 73: pp 246-254.

5. Chvapil, M. Ed. (1979) Industrial uses of Collagen. Fibrous Proteins: Scientific, Industrial and Medical Aspects, Palmerston North, New Zealand.

6. Woessner, J. F. (1976) Determination of Hydroxyproline in Connective tissues. D. A. Hall. pp 227-233.

7. Leather, R. V., M. Sisk, et al. (1994) Analysis of the collagen and total soluble nitrogen content of Isinglass finings by Polarimetry. J. Inst. Brew. 100: 331-334.

8. Marotta, M. and G. Martino (1985). “Sensitive spectrophotometric method for the quantitative estimation of collagen.” Anal. Biochem. 150: 86-90.

9. Gornall et al., (1949). Determination of serum proteins by means of the biuret reaction. J. Biol. Chem. 177: 751-766.

1. A fish skin collagen product in which native collagen forms 75% or more of the total protein content as measured by the Hofman test, which has an imino acid content less than 170 per 1000 amino acid residues, and which meets, in addition, at least one of the following requirements:
(a) in solutions of about 6 mg/mL total solids dissolved in water, more than 60% is protein recoverable from the supernatant after centrifuging at 40,000× g for 30 minutes at 5° C.; and
(b) the intristic viscosity will be between 13 and 30 dL/g determined in 0.1 M acetic acid at 10° C.

2. A collagen product as claimed in claim 1 wherein for (a) in solutions of between 2-10 mg/mL total solids dissolved in water, more than 60% is protein recoverable from the supernatant after centrifuging at 40,000× g for 30 minutes at 5° C.

3. A collagen product as claimed in claim 1 or claim 2 wherein for (a) more than 70% is protein recoverable from the supernatant after centrifuging.

4. A collagen product obtainable by stabilising a collagen paste by the addition of a preservative, said paste being the result of the following pre-stabilisation steps:
(i) without a prior extraction, effecting swelling of skins from cold water fish from which all residual flesh, fats and pigments have been removed, said swelling involving contacting said skins for an appropriate length of time with an acid solution; and
(ii) without drying said swollen skins, homogenising said skins to form a paste
each of which steps is conducted at a temperature below the thermal melting temperature (Tm) for the cold water fish species from which the skins are obtained.

5. A collagen product as claimed in claim 4 wherein the skins in step (i) are undried.

6. A collagen product as claimed in claim 4 or claim 5 wherein residual flesh, fats and pigments are removed by a mechanical cleaning process.

7. A collagen product as claimed in claim 6 wherein the mechanical cleaning process is tumble abrasion in water or uses water jetting.

8. A collagen product as claimed in any one of claims 4 to 7 wherein in step (i) the skins are contacted with said acid solution of less than 0.5 M concentration for at least 30 minutes.

9. A collagen product as claimed in any one of claims 4 to 8 wherein in step (i) the acid is selected from acetic acid, citric acid, and tartaric acid.

10. A collagen product as claimed in any one of claims 4 to 9 wherein in step (i) the skins are contacted with said acid solution at a ratio of from 10-200 g of skin per liter of acid.

11. A collagen product as claimed in any one of claims 4 to 10 wherein the homogenised skins in the form of a paste, are allowed to stand for at least 30 minutes at a temperature of less than 12° C. prior to stabilisation.

12. A collagen product as claimed in any one of claims 4 to 11 wherein said preservative is a food growth preservative.

13. A collagen product as claimed in claim 12 wherein the food growth preservative is selected from sulphite, an antimicrobial organic acid or salt thereof.

14. A collagen product as claimed in claim 13 wherein said organic acid is selected from sulphite, sorbate, acetic acid and lactic acid.

15. A collagen product as claimed in any one of claims 4 to 14 wherein said stabilised paste is dried.

16. A collagen product as claimed in any one of claims 4 to 14 wherein said paste is stored at a temperature of 15° C. or less.

17. A collagen product as claimed in claim 16 wherein said paste is stored at a temperature of 12° C. or less.

18. A method for preparing a pre-stabilised collagen paste which comprises the steps of:
(i) without a prior extraction, effecting swelling of skins from cold water fish from which all residual flesh, fats and pigments have been removed, said swelling involving contacting said skin for an appropriate length of time with an organic acid solution; and
[ii] without drying said swollen skins, homogenising said skins to form a paste,
each of which steps is conducted at a temperature below the thermal melting temperature of the cold water fish from which the skins are obtained.

19. A method for preparing a stabilised collagen paste which comprises the step of adding a preservative to a collagen paste, said paste having been obtained by the following pre-stabilisation steps:
(i) without a prior extraction, effecting swelling of skins from cold water fish from which all residual flesh, fats and pigments have been removed, said swelling involving contacting said skin for an appropriate length of time with an acid solution; and
(ii] without drying said swollen skins, homogenising said skins to form a paste,
each of which steps is conducted at a temperature below the Tm of the cold water fish from which the skins are obtained.

20. A method as claimed in claim 18 or claim 19 wherein the skins in step (i) are undried.

21. A method as claimed in claim 20 wherein the skins in step (i) are dried.

22. A method for preparing a collagen product in which at least 75% of the total protein is in the form of native collagen, said process comprising the steps of:
(i) removing all residual flesh, fat and pigments from fish skins by a mechanical cleaning process;
(ii) without a prior extraction, effecting welling of said skins through contacting said skins with an organic acid solution for an appropriate length of time;
(iii) homogenising said swollen skins to form a paste;
[iv] holding the resulting paste for an appropriate period of time to allow the collagen present to complete swelling and solubilisation.

23. A method as claimed in claim 22 wherein in step (iv), said paste is held for a minimum of 30 minutes.

24. A method as claimed in claim 21 or claim 22 wherein step (iii) is carried out prior to step (ii).

25. A method as claimed in any one of claims 22 to 24 wherein said method further includes the step of;
(v) adding a preservative to stabilise said paste.

26. A collagen product produced .by a process as claimed in any one of claims 18 to 25.

27. A collagen product as claimed in claim 26 wherein said product is in the form of a stabilised paste.

28. A collagen product as claimed in claim 26 wherein said product is a dried product.

29. A composition which includes a collagen product as claimed in any one of claims 1 to 17 and 26 to 28.

30. A composition as claimed in claim 29 wherein the composition is a foodstuff.

31. A composition as claimed in claim 29 wherein the composition is a fining agent.

32. A composition as claimed in claim 29 wherein the composition is a medicament, nutraceutical or cosmetic.

33. A method of clarifying potable liquor to remove particulate matter present in said liquor which method comprises the step of adding a collagen product as claimed in any one of claims 1 to 17 and 26 to 28, or a composition as claimed in claim 29 to said liquor.

34. A method for determining the native soluble collagen content of a protein solution, the method comprising:
(a) determining the total protein content of the solution using a biuret assay;
(b) determining the amount of native collagen in the solution using a Sirius red dye binding assay; and
(c) comparing the results of (a) and (b) to give the proportion of native collagen in the protein solution; and
wherein step (b) is carried out at a temperature less than the thermal melting temperature of the collagen being tested for.

35. A method according to claim 34 wherein the collagen being tested for is a cold water fish skin collagen.

36. A method according to claim 35 wherein step (b) is carried out at less than 20° C.

37. A kit for determining the amount of native collagen in a sample, the kit comprising copper sulphate, potassium sodium tartrate, and Sirius red.

38. A kit according to claim 37 wherein the tartrate and Sirius Red are in solution ready for use.

39. A kit according to claim 37 or claim 38 which further comprises a standard for the collagen being tested.
